(19) United States
(12) Patent Application Publication
(10) Pub. No.: US 2020/0188050 A1
(43) Pub. Date: Jun. 18, 2020

(54) ROBOTIC SURGICAL SYSTEMS AND DRAPES FOR COVERING COMPONENTS OF ROBOTIC SURGICAL SYSTEMS

(71) Applicant: Covidien LP, Mansfield, MA (US)

(72) Inventors: Raymond Pennoyer, Berlin, CT (US); Shane Reardon, Branford, CT (US)

(21) Appl. No.: 16/638,889

(22) PCT Filed: Aug. 16, 2018
(86) PCT No.: PCT/US2018/000289

§ 371 Date: Feb. 13, 2020

Related U.S. Application Data
(60) Provisional application No. 62/546,201, filed on Aug. 16, 2017.

(51) Int. Cl. A61B 46/10; A61B 34/30; A61B 50/13

(52) U.S. Cl. CPC A61B 46/10; A61B 2017/00477; A61B 50/13; A61B 34/30

(57) ABSTRACT

A drape for covering a robotic surgical system includes a body having a first end portion and a second end portion. The second end portion has a proximal opening. The body includes a cavity extending between the first end portion and the second end portion thereof. The cavity is dimensioned for receipt of a portion of the robotic surgical system via the proximal opening of the second end portion of the body. The drape includes a deployment handle coupled to the second end portion of the body at a position distal of the proximal opening. The deployment handle is configured to facilitate positioning of the body of the drape adjacent the robotic surgical system such that the portion of the robotic surgical system is disposed within the cavity of the body

### BACKGROUND

Robotic surgical systems have been used in minimally invasive medical procedures. Some robotic surgical systems include a console supporting a surgical robotic arm and a surgical instrument, having at least one end effector (e.g., forceps or a grasping tool), mounted to the surgical robotic arm. The surgical robotic arm provides mechanical power to the surgical instrument for its operation and movement.

In robotic assisted medical procedures, the various components of a robotic surgical system are generally draped to decrease the probability of inadvertent contamination of an external surgical sterile field. Accordingly, it would be beneficial to provide a means for more easily deploying a drape while decreasing the probability of inadvertent contamination of the external surgical sterile field.

### SUMMARY

In accordance with an aspect of the present disclosure, a drape for covering a robotic surgical system is provided. The drape includes a body having a first end portion and a second end portion. The second end portion has a proximal opening. The body includes a cavity extending between the first end portion and the second end portion thereof. The cavity is dimensioned for receipt of a portion of the robotic surgical system via the proximal opening of the second end portion of the body. The drape includes a deployment handle coupled to the second end portion of the body at a position distal of the proximal opening. The deployment handle is configured to facilitate positioning of the body of the drape adjacent the robotic surgical system such that the portion of the robotic surgical system is disposed within the cavity of the body.

In some embodiments, the body of the drape may include an outer surface and an inner surface. The deployment handle may be fixed to the outer surface of the body of the drape.

It is contemplated that the second end portion of the body of the drape may include a proximal-most end having a proximal edge. The deployment handle may be fixed to the outer surface of the body, distal of the proximal edge at the proximal-most end of the body of the drape.

It is envisioned that the deployment handle may include a body having a first portion and a second portion. The first portion and the second portion of the deployment handle may be spaced apart and coupled to the outer surface of the body of the drape such that a handle loop is formed therebetween. The handle loop may be dimensioned to receive a hand of a clinician to facilitate positioning of the portion of the robotic surgical system within the cavity of the drape via the proximal opening of the drape.

In another aspect of the present disclosure, a method of draping a robotic surgical system is provided. The method includes positioning a drape, in a folded form, adjacent the robotic surgical system. The drape includes a body having a distal opening, a proximal opening, and a cavity extending along a length of the body. The distal opening has a rigid ring and the proximal opening has a proximal edge. The drape also includes a deployment handle disposed on the body of the drape at a position distal of the proximal edge of the proximal opening. The method further includes locating a surgical assembly of the robotic surgical system within the proximal opening of the drape via the deployment handle such that the surgical assembly is disposed within the cavity of the body adjacent the proximal opening.

In some embodiments, the method may include advancing the drape, in the folded form, over a slide rail and an instrument drive unit of the surgical assembly until a distal end of the instrument drive unit is aligned with the rigid ring of the drape, and fixing the distal end of the instrument drive unit with the rigid ring of the drape.

It is contemplated that the method may include connecting a sterile interface module of the surgical assembly to the distal end of the instrument drive unit such that the rigid ring of the drape is disposed between the distal end of the instrument drive unit and the sterile interface module.

It is envisioned that the method may further include maneuvering the drape proximally, via the deployment handle, such that the drape is telescopically unfolded from the folded form of the drape to a telescopically unfolded form of the drape, and a surgical robotic arm of the robotic surgical system is disposed within the cavity of the body of the drape.

In some embodiments, the method may include coupling a surgical instrument of the robotic surgical system with the sterile interface module of the surgical assembly.

In yet another aspect of the present disclosure, a robotic surgical system is provided and includes a surgical robotic arm, a surgical assembly coupled to the surgical robotic arm, and a drape. The drape includes a body having a distal opening, a proximal opening, and a cavity extending along a length of the body. The distal opening has a rigid ring and the proximal opening has a proximal edge. The cavity is dimensioned for receipt of the surgical robotic arm and the surgical assembly via the proximal opening of the body. The drape includes a deployment handle coupled to the body at a position distally adjacent of the proximal edge of the proximal opening. The deployment handle is configured to facilitate positioning of the body of the drape adjacent the robotic surgical system such that the surgical robotic arm and the surgical assembly of the robotic surgical system are disposed within the cavity of the body of the drape.

In some embodiments, the body of the drape may include a first end portion and a second end portion. The second end portion may have a proximal-most end. The deployment handle may be fixed to an outer surface of the body, distally adjacent the proximal-most end of the body.

In accordance with another aspect of the present disclosure, a surgical drape system for covering a robotic surgical system is provided. The surgical drape system includes a first drape, a second drape, and a third drape.

The first drape includes a body having a first end portion, a second end portion, and a cavity extending between the first end portion and the second end portion thereof. The cavity of the first drape is dimensioned for receipt of a first portion of the robotic surgical system.

The first drape further includes a first deployment handle coupled to the second end portion of the body. The first deployment handle is configured to facilitate positioning of the body of the first drape adjacent the robotic surgical system such that the first portion of the robotic surgical system is disposed within the cavity of the body of the first drape.

The second drape includes a body having a first end portion, a second end portion, and a cavity extending between the first end portion and the second end portion thereof. The cavity of the second drape is dimensioned for receipt of a second portion of the robotic surgical system.

The second drape further includes a second deployment handle coupled to the second end portion of the body. The deployment handle is configured to facilitate positioning of the body of the second drape adjacent the robotic surgical system such that the second portion of the robotic surgical system is disposed within the cavity of the body of the second drape.

The third drape includes a body having a first end portion, a second end portion, and a cavity extending between the first end portion and the second end portion thereof. The cavity of the third drape is dimensioned for receipt of a third portion of the robotic surgical system.

Further details and aspects of exemplary embodiments of the present disclosure are described in more detail below with reference to the appended figures.

### BRIEF DESCRIPTION OF THE DRAWINGS

Embodiments of the present disclosure are described herein with reference to the accompanying drawings, wherein:

FIG. 1 is a schematic illustration of a robotic surgical system including a robotic surgical assembly in accordance with the present disclosure;

FIG. 2 is a perspective view of the robotic surgical assembly of FIG. 1 attached to a surgical robotic arm, which is attached to a robotic arm cart;

FIGS. 3A is a perspective view of a drape of the robotic surgical system of FIG.1 covering a portion of a surgical robotic arm of the robotic surgical assembly of FIG. 1;

FIGS. 3B is a perspective view of the drape of FIG. 3A covering a further portion of the robotic arm cart of the robotic surgical assembly of FIG. 1;

FIGS. 3C is a perspective view of the drape of FIG. 3A covering still a further portion of the robotic arm cart of the robotic surgical assembly of FIG. 1;

FIG. 4A is a top view of the drape of FIG. 1 in a folded form;

FIG. 4B is a perspective view of the drape of FIG. 1 in a telescopically unfolded form;

FIG. 4C is a perspective view of the area of detail indicated as “4C” in FIG. 4B illustrating components of an intermediate portion of the drape of FIG. 1;

FIG. 5A is a perspective view of a rigid ring of the drape of FIG. 4B;

FIG. 5B is a front, top view of the rigid ring of FIG. 5A;

FIG. 5C is a front, bottom view of the rigid ring of FIG. 5A;

FIG. 5D is a side view of the rigid ring of FIG. 5A;

FIG. 5E is a longitudinal, cross-sectional view taken across section line “5E-5E” of FIG. 5C;

FIG. 5F is an enlarged view of the area of detail indicated as “5F” in FIG. 5E;

FIG. 6A is a perspective view of a mounting sheath of the drape of FIG. 4B;

FIG. 6B is a top view of the mounting sheath of FIG. 6A;

FIG. 6C is a bottom view of the mounting sheath of FIG. 6A;

FIG. 6D is a back view of the mounting sheath of FIG. 6A;

FIG. 6E is a side view of the mounting sheath of FIG. 6A;

FIG. 6F is a front view of the mounting sheath of FIG. 6A;

FIG. 7A is a top, perspective view of a deployment handle of the drape of the robotic surgical system of FIG.1;

FIG. 7B is a top, perspective view of the deployment handle of FIG. 3, illustrating a configuration of the deployment handle in use; and

FIG. 8 is a perspective view of a surgical drape system of the robotic surgical system of FIG.1, in accordance with another embodiment of the present disclosure, illustrating a first drape, a second drape, and a third drape covering respective portions of the surgical robotic arm and the robotic arm cart of the robotic surgical assembly of FIG. 1.

### DETAILED DESCRIPTION

Embodiments of the presently disclosed robotic surgical system including a robotic arm cart, a surgical robotic arm, a surgical assembly (including an instrument drive unit (“IDU”) and a surgical instrument), and a drape for covering some or all of the aforementioned components, are described in detail with reference to the drawings, in which like reference numerals designate identical or corresponding elements in each of the several views. As used herein the term “distal” refers to that portion of the robotic arm cart, surgical robotic arm, surgical assembly, or drape, that is closer to the patient, while the term “proximal” refers to that portion of the robotic arm cart, surgical robotic arm, surgical assembly, or drape, that is farther from the patient.

As will be described in detail below, provided is a drape for covering various components of a robotic surgical system. The drape maintains an external surgical sterile field by sheathing or encapsulating the surgical assembly disposed therein. The drape includes a deployment handle to facilitate deployment of the drape by a sterile clinician while decreasing the probability of accidental contamination of the sterile clinician and/or the external surgical sterile field.

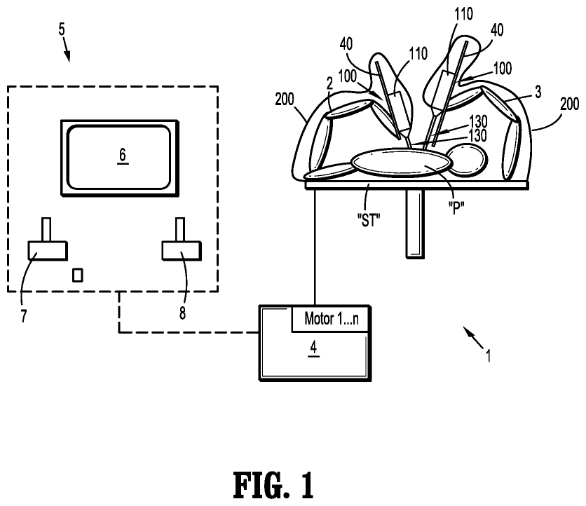

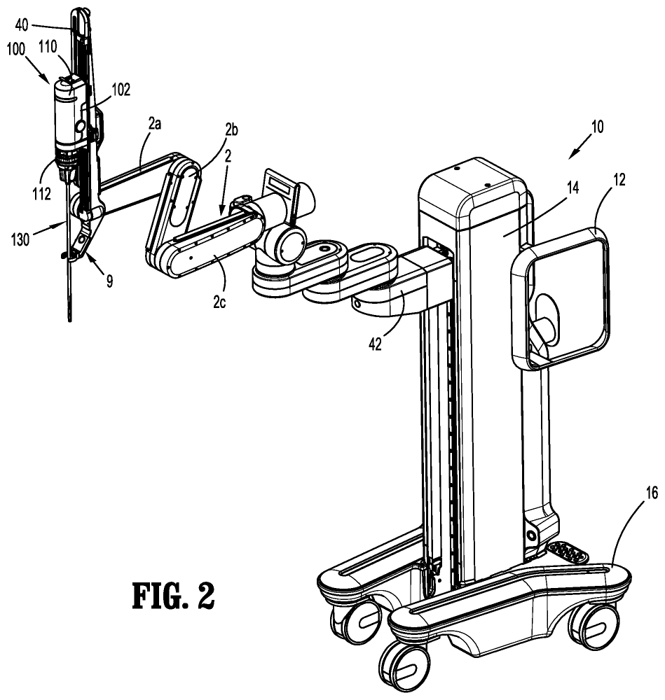

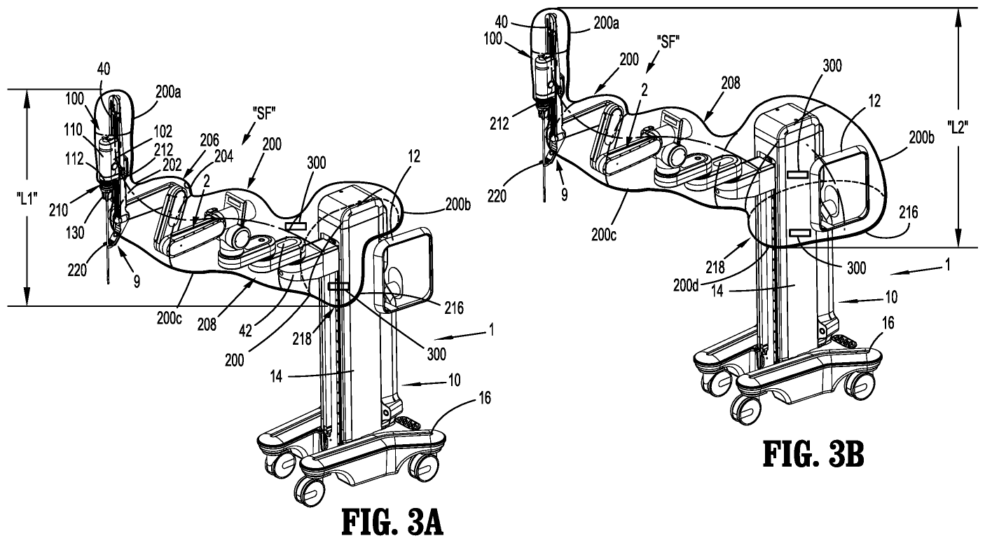

Referring initially to FIGS. 1 and 2, a surgical system, such as, for example, a robotic surgical system 1, generally includes a surgical robotic arm or surgical robotic arms 2, 3 coupled to a robotic arm cart 10, a surgical assembly 100 coupled to the surgical robotic arm 2, and a drape 200 for covering the surgical robotic arms 2 (FIGS. 3A-3C), 3 and the surgical assembly 100. In some embodiments, the drape 200 may be dimensioned to also cover the robotic arm cart 10. The surgical assembly 100 includes an instrument drive unit (hereinafter “IDU”) 110 coupled to a slide rail 40 of surgical robotic arms 2, 3, and an electromechanical surgical instrument 130 operably coupled to IDU 110 by a sterile interface module 112 of surgical assembly 100.

In some embodiments, the robotic surgical system 1 may include a mount assembly 9 (see FIGS. 2-3C) coupled to the surgical robotic arm 2 and configured to releasably couple a surgical accessory such as, for example a trocar, a surgical port, an optical device, or the like to the surgical robotic arm 2. It is contemplated that the mount assembly 9 may add to the stability to the electromechanical surgical instrument 130 passed therethrough. During a surgical procedure, the electromechanical surgical instrument 130 may undergo undesired reaction loading as a result of forces exerted upon the electromechanical surgical instrument 130 by a natural orifice or the surrounding tissue of an incision, such as, for example, an incision through an abdominal wall. By utilizing the mount assembly 9 and a surgical port, the mount assembly 9 may assist in inhibiting the transfer of such forces upon the electromechanical surgical instrument 130 minimizing instrument deflection. For a detailed discussion of the construction and operation of a mount assembly, reference may be made to U.S. Patent Application No. TBD, entitled “SYSTEMS AND ASSEMBLIES FOR MOUNTING A SURGICAL ACCESSORY TO ROBOTIC SURGICAL SYSTEMS, AND PROVIDING ACCESS THERETHROUGH,” (Attorney Docket: C00014354.USP1 (203-11477)), the entire contents of which are incorporated by reference herein.

The robotic surgical system 1 further includes a control device 4 and an operating console 5 coupled with control device 4. Operating console 5 includes a display device 6, which is set up in particular to display three-dimensional images; and manual input devices 7, 8, by means of which a person (not shown), for example a clinician, is able to telemanipulate surgical robotic arms 2, 3 in a first operating mode, as known in principle to a person skilled in the art. Each of the surgical robotic arms 2, 3 may be composed of a plurality of members 2a, 2b , 2c, which are connected through joints. Surgical robotic arms 2, 3 may be driven by electric drives (not shown) that are connected to control device 4. Control device 4 (e.g., a computer) may be set up to activate the drives, in particular by means of a computer program, in such a way that surgical robotic arms 2, 3, the attached robotic surgical assembly 100, and thus electromechanical surgical instrument 130 (including an electromechanical end effector (not shown)) execute a desired movement according to a movement defined by means of manual input devices 7, 8. Control device 4 may also be set up in such a way that it regulates the movement of surgical robotic arms 2, 3.

Robotic surgical system 1 is configured for use on a patient “P” lying on a surgical table “ST” to be treated in a minimally invasive manner by means of a surgical instrument, e.g., electromechanical surgical instrument 130. In embodiments, surgical robotic arms 2, 3 may be coupled to robotic arm cart 10 (FIG. 2) rather than surgical table “ST.” Robotic surgical system 1 may also include more than two surgical robotic arms 2, 3, the additional surgical robotic arms likewise being connected to control device 4 and being telemanipulatable by means of operating console 5. A surgical instrument, for example, electromechanical surgical instrument 130 (including the electromechanical end effector), may also be attached to the additional surgical robotic arm.

Control device 4 may control a plurality of motors, e.g., motors (Motor 1 . . . n), with each motor configured to drive movement of surgical robotic arms 2, 3 in a plurality of directions. Further, control device 4 may control a motor assembly (not shown) of IDU 110 of robotic surgical assembly 100 that drives various operations of surgical instrument 130.

For a detailed discussion of the construction and operation of a robotic surgical system, reference may be made to U.S. Pat. No. 8,828,023, entitled “MEDICAL WORKSTATION,” the entire contents of which are incorporated by reference herein.

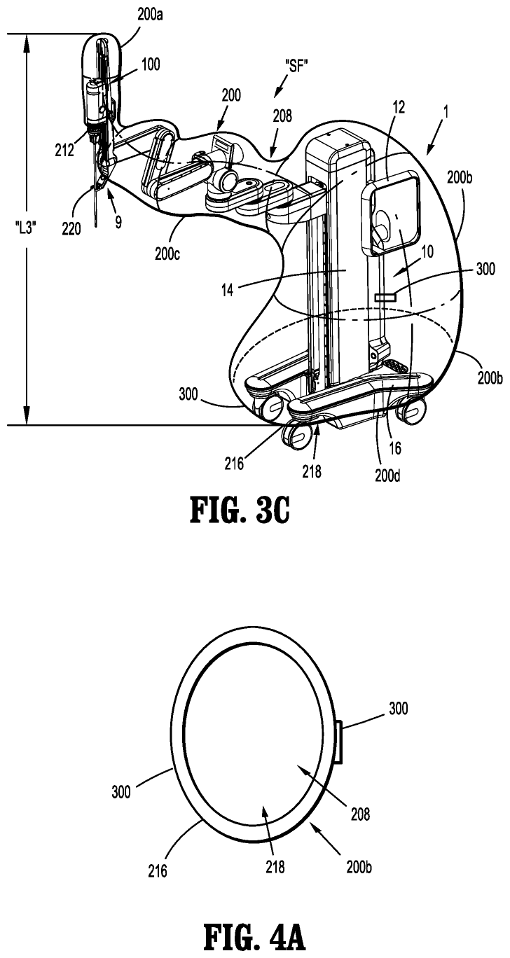

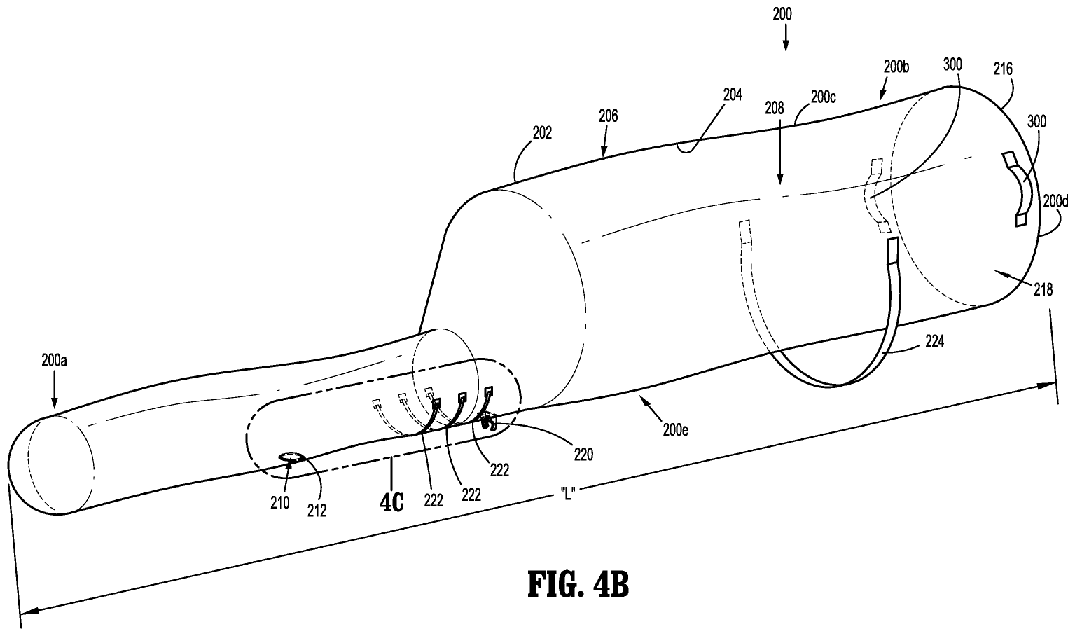

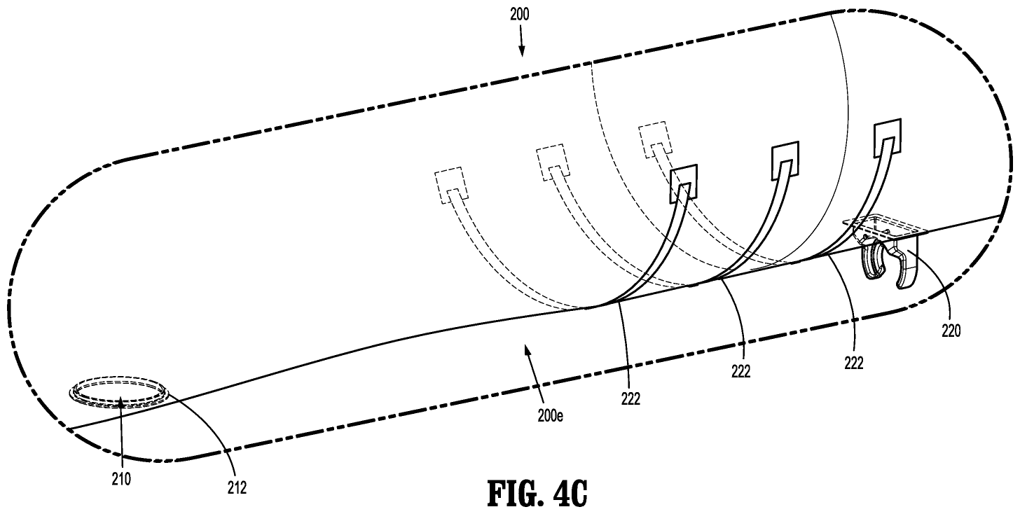

With reference to FIGS. 3A-6, drape 200 of robotic surgical system 1 has a generally elongated configuration, such as, for example, a tubular shape (see FIG. 4B), and is fabricated from a resilient material, such as, for example, a natural and/or synthetic fabric or layered material that is impermeable to liquids/moisture. Drape 200 may, in embodiments, be a single layer or a laminate, and may be made of, e.g., a nonwoven spun bonded olefin fiber material known as TYVEK®, which is vapor/gas permeable, fluid-resistive, and prevents microbes from passing therethrough. In other embodiments, drape 200 may be made of low-density polyethylene (LDPE), high-density polyethylene (HDPE), polypropylene, polyurethane, and/or polyethylene materials or other similar non-toxic, biocompatible compounds. In some embodiments, only some portions of drape 200 may be fabricated from ^{TYVEK}® and at various locations of drape 200. Drape 200 may be translucent so that the components of surgical assembly 100 that drape 200 covers remain visible to a clinician. It is contemplated that drape 200 may be opaque rather than translucent or opaque and translucent along varying portions. In some embodiments, a high density polyethylene spun woven fiber or synthetic fabric may be glued, thermally bonded, ultrasonically welded, stitched, Velcro®, or seam bonded onto drape 200.

In embodiments, drape 200 may be received by the clinician in a telescopically folded form, as illustrated in FIG. 4A, and may include a telescopically unfolded form, as illustrated in FIG. 4B. Drape 200 has a body 200c including a first end portion or distal end portion 200a, and a second end portion or proximal end portion 200b.

The body 200c of drape 200 may have any length “L” (see FIG. 4B) suitable to cover various portions of the robotic surgical system 1. For example, as shown in FIG. 3A, drape 200 may have a sufficient length “L1” to at least allow for second end portion 200b of drape 200 to fit over a base or proximal portion 42 of surgical robotic arm 2. As shown in FIG. 3B, drape 200 may have a sufficient length “L2” to at least allow for second end portion 200b thereof to fit over a handle portion 12 of robotic arm cart 10 and to be secured to a post 14 of robotic arm cart 10. As shown in FIG. 3C, drape 200 may have a sufficient length “L3” to at least allow for second end portion 200b thereof to fit over a base 16 of robotic arm cart 10. Drape 200 may have a length to accommodate surgical robotic arm 2 in a fully extended position. It is also contemplated that drape 200 may have any suitable width to cover various portions of the robotic surgical system 1. As shown in FIG. 4B, in one embodiment, body 200c of drape 200 may include varying suitable widths to cover various portions of the robotic surgical system 1.

With continued reference to FIGS. 3A-3C, drape 200 of robotic surgical system 1 is defined by a drape wall 206 having an outer surface 202 and an inner surface 204. Drape wall 206 may be fabricated from the same or a single material and be monolithically formed, or, in some embodiments, drape wall 206 may be fabricated from layers of different materials or from the same material having different properties. Drape 200 defines a cavity 208 therein extending along the length “L” between the first end portion 200a and the second end portion 200b of body 200c. Cavity 208 is configured to sheath or encapsulate portions of robotic surgical system 1 from a surgical sterile field “SF” outside of drape wall 206 of drape 200. In embodiments, cavity 208, adjacent the first end portion 200a, is dimensioned to receive, sheath, or encapsulate surgical assembly 100 (e.g., instrument drive unit 110 and slide rail 40). First end portion 200a is sealed such that surgical assembly 100 is prohibited from extending distally beyond first end portion 200a.

With continued reference to FIGS. 3A-3C and additional reference to FIG. 4B, drape 200 includes an intermediate portion 200e disposed between first end portion 200a and second end portion 200b of drape 200. Intermediate portion 200e includes a first or distal opening 210 defined therein. In one embodiment, distal opening 210 may be disposed substantially adjacent to or in close proximity to the first end portion 200a. In alternative embodiments, distal opening 210 may be disposed in any suitable portion of body 200c of drape 200. Distal opening 210 is in fluid communication with cavity 208 of body 200c. Distal opening 210 has a generally circular or annular shape dimensioned to form a fluid-resistant seal with sterile interface module 112 of surgical assembly 100.

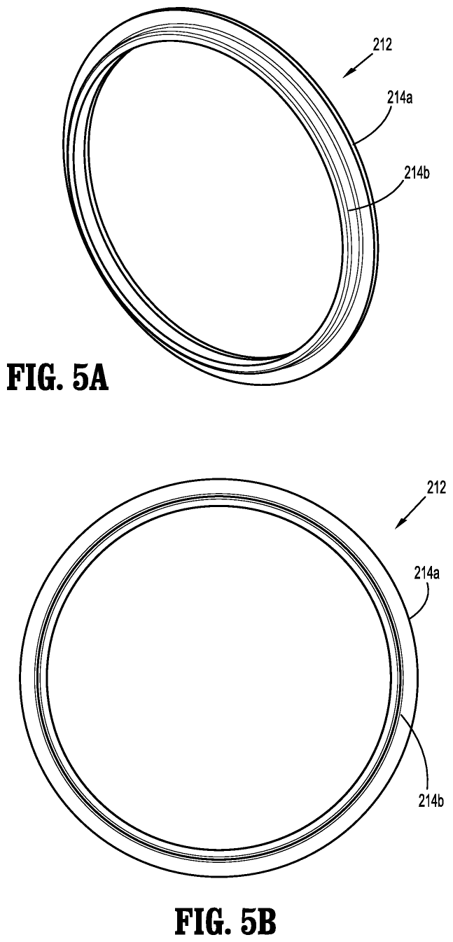

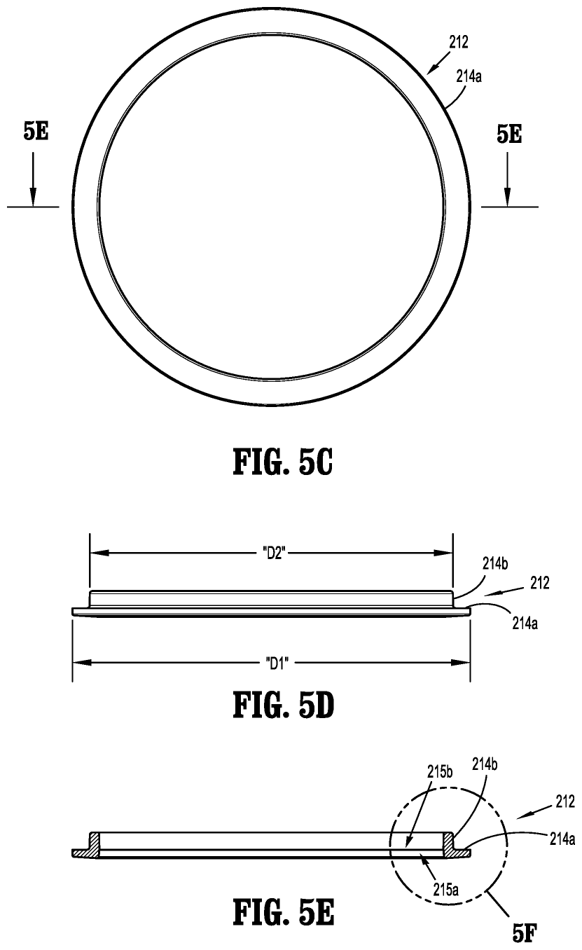

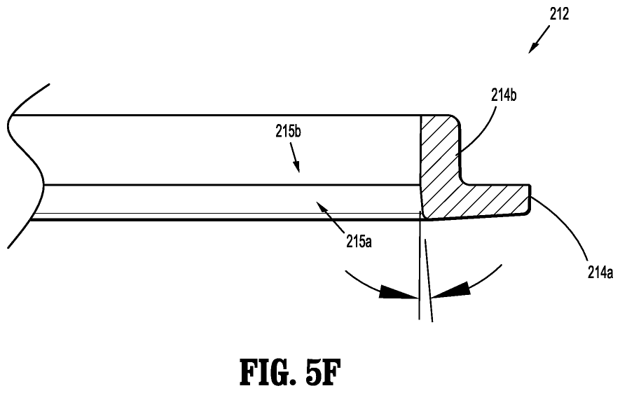

With continued reference to FIGS. 3A-3C and additional reference to FIGS. 5A-5E, during assembly, drape 200 is placed over rail 40 and instrument drive unit 110, and sterile interface module 112 is positioned to extend through distal opening 210 of drape 200 with surgical instrument 130, connected to, and extending from, sterile interface module 112. Distal opening 210 of drape 200 includes a rigid ring 212. Rigid ring 212 includes a flange portion 214a, having an outer diameter “D1,” and a cuff portion 214b extending distally from flange portion 214a, having an outer diameter “D2” that is less than outer diameter “D1” of flange portion 214a. Flange portion 214a defines a chamfered inner surface 215a (FIG. 5F) proximally adjacent an inner surface 215b (FIG. 5F) of cuff portion 214b. Rigid Ring member 212 is configured to be disposed between the instrument drive unit 110 and the sterile interface module 112, and to enable operative interconnection between the instrument drive unit 110 and the sterile interface module 112. Rigid ring 212 may be secured to a distal portion of the instrument drive unit 110 such that, the distal portion of the instrument drive unit 110 engages inner surface 215b of cuff portion 214b. Further, rigid ring 212 may be secured to a proximal portion of the sterile interface module 112 such that, the proximal portion of the sterile interface module 112 engages chamfered inner surface 215a of flange portion 214a. In embodiments, rigid ring 212 may be secured to instrument drive unit 110 and sterile interface module 112 via a unilateral axial attachment (e.g., push-in, snap-fit, and/or loose-fit type arrangement) such that, sterile interface module 112 is allowed to rotate relative to and within distal opening 210 of drape 200 while maintaining sterile interface module 112 axially fixed therein.

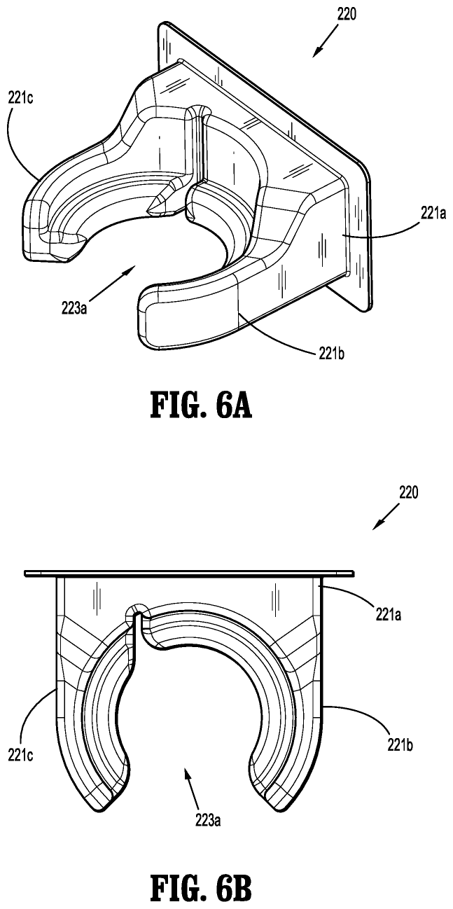

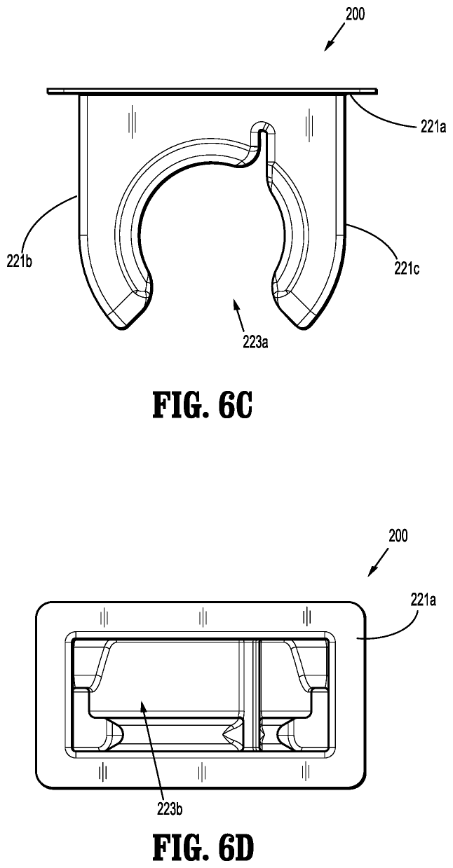

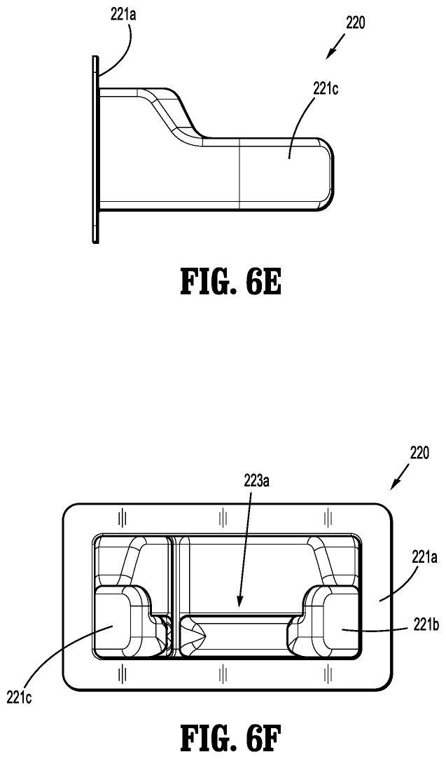

With additional reference to FIGS. 6A-6F, in some embodiments, intermediate portion 200e of drape 200 may include a jaw mounting sheath or sleeve 220 disposed/formed on the body 200c of drape 200 proximal of the distal opening 210. Alternatively, mounting sheath 220 may be disposed/formed on any suitable portion of body 200c of drape 200. Mounting sheath 220 includes a body portion 221a, a first arm 221b extending from a first side of body portion 221a, and a second arm 221c extending from a second side of body portion 221a. First and second arms 221b, 221c are spaced apart to define a mounting channel 223a theretween. As shown in FIG. 6D, mounting sheath 220 includes a sheath cavity 223b defined within, and in collaboration with, body portion 221a, first arm 221b, and second arm 221c. Sheath cavity 223b of mounting sheath 220 is configured to receive and enshroud or enclose all of or a portion of mount assembly 9 (see FIGS. 2-3C). It is contemplated that the mounting sheath 220 may be positioned and maintained between mount assembly 9 and the surgical accessory coupled thereto. The mounting sheath 220 is configured to work collaboratively with drape 200 to minimize obstruction of the surgical sterile field “SF” by drape 200 and to provide expeditious coupling and uncoupling of the surgical accessory to the surgical robotic arm 2, via mount assembly 9. For a detailed discussion of the construction and use of mounting sheath 220, reference may once again be made to U.S. Patent Application No. TBD, entitled “SYSTEMS AND ASSEMBLIES FOR MOUNTING A SURGICAL ACCESSORY TO ROBOTIC SURGICAL SYSTEMS, AND PROVIDING ACCESS THERETHROUGH,” (Attorney Docket: C00014354.USP1 (203-11477)), the entire content of which was previously incorporated by reference herein.

With continued reference to FIGS. 3A-3C, the cavity 208 of body 200c between first end portion 200a and second end portion 200b is dimensioned to sheath, encapsulate, or house elongate members 2a, 2b, 2c of surgical robotic arm 2. Cavity 208 of body 200c, adjacent the second end portion 200b, is dimensioned to receive, sheath, or encapsulate at least proximal portion 42 of surgical robotic arm 2 and/or a portion or portions of robotic arm cart 10. Inner surface 204 of drape 200 extends to a proximal edge 216 of drape 20 and defines a second or proximal opening 218 at the second end portion 200b of drape 200. As such, proximal opening 218 of second end portion 200b of drape 200 is in fluid communication with cavity 208 of body 200c of drape 200. Proximal opening 218 of drape 200 has a generally circular or annular shape and is dimensioned to fit over handle portion of robotic arm cart 10 and/or robotic arm cart 10, and in embodiments, over the entirety of handle portion of robotic arm cart 10. Proximal edge 216 and proximal opening 218 of second end portion 200b of drape 200 may be located at a proximal-most end 200d of drape 200 rather than a side of drape 200 as is distal opening 210 of intermediate portion 200e. As such, drape 200 is open at its proximal-most end 200d, whereas drape 200 is closed at its distal-most end. Alternatively, it is contemplated that proximal opening 218 may be located anywhere along a length of drape 200.

It is contemplated that inner surface 204 of drape 200 adjacent the proximal opening 218 of drape 200 may include an adhesive lining (not shown) disposed/formed on an inner periphery thereof for fixing second end portion 200b of drape 200 to robotic arm cart 10. In an embodiment, inner surface 204 of drape 200, adjacent the proximal opening 218 of drape 200, may include an elastic band (not explicitly shown), Velcro®, cinch line, bungee hooks, magnetic material, or the like, surrounding a periphery of second end portion 200b to assist in securing robotic arm cart 10 within second end portion 200b. In some embodiments, instead of inner surface 204 of drape 200, adjacent the proximal opening 218, having an elastic band, proximal edge 216 may have a tie cord (not explicitly shown) disposed adjacent proximal opening 218 to allow for the diameter of proximal opening 218 to be adjusted to fit over and secure to various portions of robotic arm cart 10.

With continued reference to FIGS. 3A-3C and additional reference to FIGS. 4B and 4C, in some embodiments the intermediate portion 200e may include a plurality of first attachment members 222 disposed/formed on the body 200c of drape 200 between the distal opening 210 and the mounting sheath 220. Alternatively, the plurality of first attachment members 222 may be disposed/formed on any suitable portion of body 200c of drape 200. The plurality of first attachment members 222 may include a deformable band, elastic band, Velcro®, cinch line, bungee hooks, magnetic material, or the like, surrounding a periphery or a portion of the periphery of drape wall 206 adjacent the plurality of first attachment members 222 to releasably secure the drape wall 206 to surgical robotic arm 2.

Similarly, the intermediate portion 200e may include a second attachment member 224 disposed/formed on the body 200c of drape 200 between the mounting sheath 220 and second end portion 200b, or alternatively on any suitable portion of body 200c of drape 200. The second attachment feature 224 may include a deformable band, elastic band, Velcro®, cinch line, bungee hooks, magnetic material, or the like, surrounding a periphery or a portion of the periphery of drape wall 206 adjacent the second attachment member 224 to releasably secure the drape wall 206 to robotic arm cart 10.

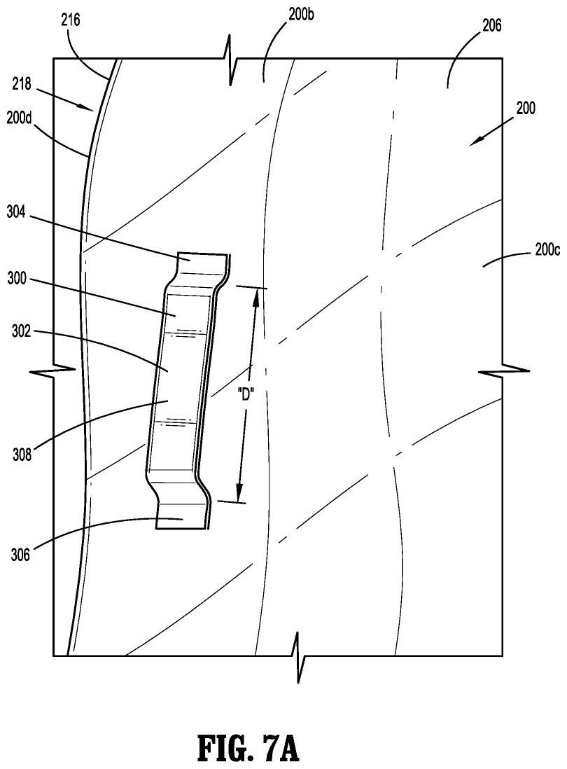

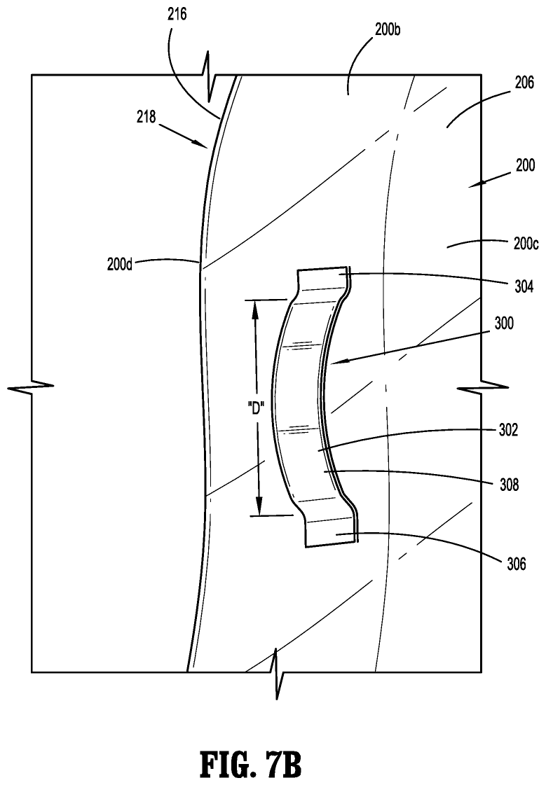

With continued reference to FIGS. 3A-4B and additional reference to FIGS. 7A and 7B, drape 200 includes a deployment handle 300 configured to facilitate positioning and maneuvering of drape 200 for covering surgical assembly 100, surgical robotic arm 2, and/or robotic arm cart 10. In embodiments, deployment handle 300 is coupled to second end portion 200b of body 200c of drape 200. In one embodiment, deployment handle 300 may be coupled to second end portion 200b of body 200c of drape 200 at a location distally adjacent the proximal-most end 200d of drape 200, such as, for example, distally adjacent proximal edge 216 adjacent proximal opening 218. Alternatively, deployment handle 300 may be disposed/formed at any location along the length of body 200c of drape 200.

With reference to FIGS. 7A and 7B, deployment handle 300 includes a body 302 having a first portion 304 and an opposing or second portion 306. First and second portions 304, 306 of body 302 of deployment handle 300 are spaced apart a distance “D” and fixed or formed to outer surface 202 of drape wall 206 of drape 200 such that a handle loop 308 is formed between first and second portions 304, 306 of deployment handle 300. In embodiments, first and second portions 304, 306 of deployment handle 300 may be fixed to outer surface 202 of drape 200 using adhesives, stitching, welding, or the like. Alternatively, any suitable method of fixing first and second portions 304, 306 of deployment handle 300 to outer surface 202 of drape 200 may be utilized. In other embodiments, first and second portions 304, 306 of deployment handle 300 may be removably fixed to outer surface 202 of drape 200 using Velcro®, 3M® double sided tape, or the like.

With continued reference to FIGS. 7A and 7B, it is contemplated that handle loop 308, between first and second portions 304, 306 of deployment handle 300, is dimensioned to accommodate a width of a clinician's hand. In embodiments, handle loop 308 of deployment handle 300 may be flexible to accommodate various hand sizes and may be ergonomically shaped to facilitate grasping by a clinician with either the left or right hand. Additionally, drape 200 may include more than one deployment handle 300 disposed/formed on outer surface 202 of drape 200 at various locations (e.g., opposing sides of drape wall 206 of drape 200), as illustrated in FIGS. 3A-3C. Deployment handle 300 of drape 200 is fabricated from a resilient material, such as, for example, a natural and/or synthetic fabric or layered material similar to or the same as body 200c of drape 200. In one embodiment, deployment handle 300 may be fabricated from a high density polyethylene material or the like.

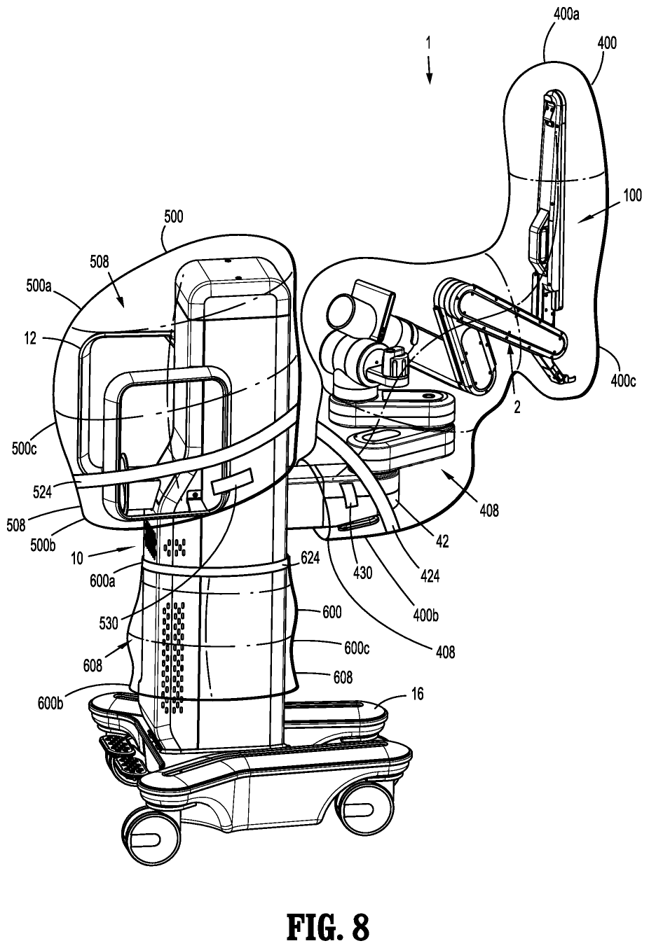

Turning briefly to FIG. 8, in embodiments, robotic surgical system 1 may include a surgical drape system having a plurality of drapes, such as, for example, a first drape 400, a second drape 500, and a third drape 600.

First drape 400 is substantially similar to drape 200 as described above. First drape 400 includes a body 400c having a first end portion 400a, a second end portion 400b, and a cavity 408 extending between first and second end portions 400a, 400b. First drape 400 is sized and configured to at least allow for second end portion 400b of first drape 400 to fit over proximal portion 42 of surgical robotic arm 2 to sheath or encapsulate portions of proximal portion 42 of surgical robotic arm 2 within cavity 408 of first drape 400.

Second drape 500 includes a body 500c having a first end portion 500a, a second end portion 500b, and a cavity 508 extending between first and second end portions 500a, 500b. Second drape 500 is sized and configured to at least allow for second end portion 500b thereof to fit over handle portion 12 of robotic arm cart 10 to sheath or encapsulate portions of handle portion 12 within cavity 508 of second drape 500.

Third drape 600 includes a body 600c having a first end portion 600a, a second end portion 600b, and a cavity 608 extending between first and second end portions 600a, 600b. Third drape 600 is sized and configured to at least allow for a second end portion 600b thereof to fit over a portion of robotic arm cart 10 adjacent base 16 of robotic arm cart 10 to sheath or encapsulate portions of robotic arm cart 10 adjacent base 16 within cavity 608 of third drape 600. It is contemplated that third drape 600 may initially be provided as a single sheet of drape material which is configured to be fold or wrapped around the portion of robotic arm cart 10 adjacent base 16, and secured thereto, to define a substantially skirt-like configuration.

In embodiments, each of first and second drapes 400, 500, includes a deployment handle 430, 530, respectively, similar to deployment handle 300 of drape 200, as described above. Further, in embodiments, each of first, second, and third drapes 400, 500, 600 includes an attachment feature 424, 524, 624, respectively, similar to second attachment feature 224, as described above. Specifically, attachment features 424, 524, 624 may include a deformable band, elastic band, Velcro®, cinch line, bungee hooks, magnetic material, double sided tape, or the like, surrounding a periphery or a portion of the periphery of the respective first, second, and third drapes 400, 500, 600 to releasably secure the first, second, and third drapes 400, 500, 600 to portions of the surgical robotic arm 2 and/or the robotic arm cart 10.

With reference to FIGS. 1-7B, in operation, proximal portion 42 of surgical robotic arm 2 is coupled to robotic arm cart 10, and instrument drive unit 110 is coupled to slide rail 40 of surgical robotic arm 2.

Drape 200 may now be positioned over instrument drive unit 110, surgical robotic arm 2, and/or a portion or portions of robotic arm cart 10. In embodiments, drape 200 may be received by the clinician in a telescopically folded form, as illustrated in FIG. 4A. Folded drape 200 is then placed over instrument drive unit 110 and slide rail 40 by inserting slide rail 40 and instrument drive unit 110 into proximal opening 218 and into cavity 208 of drape 200. Folded drape 200 is advanced over slide rail 40 and instrument drive unit 110 until a distal end of instrument drive unit 110 is aligned with and connected to rigid ring 212 of drape 200. With rigid ring 212 connected to the distal end of instrument drive unit 110, sterile interface module 112 is then coupled to the distal end of instrument drive unit 110 thereby sandwiching rigid ring 212 of drape 200 between instrument drive unit 110 and sterile interface module 112.

Then, using handle loop 308 of deployment handle 300, second end portion 200b of drape 200 is pulled axially in a proximal direction to position surgical assembly 100, including slide rail 40, and elongate members 2a, 2b, 2c of surgical robotic arm 2 within cavity 208 of drape 200. Proximal movement of drape 200 along surgical robotic arm 2 is continued until the elastic band, draw string, Velcro®, draw string, cinch line, bungee hooks, magnetic material, or the like, of second end portion 200b of drape 200 is passed over handle portion 12 of robotic arm cart 10, thereby disposing handle portion 12 of robotic arm cart 10 within cavity 208 adjacent second end portion 200b of drape 200.

With drape 200 covering each of instrument drive unit 110, surgical robotic arm 2, and handle portion 12 of robotic arm cart 10, surgical instrument 130 may be attached to sterile interface module 112.

Accordingly, surgical assembly 100, surgical robotic arm 2, and handle portion 12 of robotic arm cart 10 are sheathed or encapsulated within cavity 208 of drape 200 and prevented from inadvertently contaminating the sterile clinician, surgical instrument 130, and/or the surgical sterile field “SF.” Upon completion of the medical procedure, surgical instrument 130 and sterile interface module 112 may be detached from instrument drive unit 110, and drape 200 may be removed using handle loop 308 of deployment handle 300.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A drape for covering a robotic surgical system, the drape comprising:
a body including a first end portion and a second end portion having a proximal opening, the body having a cavity extending between the first end portion and the second end portion thereof, the cavity dimensioned for receipt of a portion of the robotic surgical system via the proximal opening of the second end portion of the body; and
a deployment handle coupled to the second end portion of the body at a position distal of the proximal opening, the deployment handle configured to facilitate positioning of the body of the drape adjacent the robotic surgical system such that the portion of the robotic surgical system is disposed within the cavity of the body.

2. The drape according to claim 1, wherein the body of the drape has an outer surface and an inner surface, wherein the deployment handle is fixed to the outer surface of the body.

3. The drape according to claim 2, wherein the second end portion of the body includes a proximal-most end having a proximal edge, wherein the deployment handle is fixed to the outer surface of the body, distal of the proximal edge at the proximal-most end of the body.

4. The drape according to claim 2, wherein the deployment handle includes a body having a first portion and a second portion spaced apart and coupled to the outer surface of the body such that a handle loop is formed therebetween, the handle loop dimensioned to receive a hand of a clinician to facilitate positioning of the portion of the robotic surgical system within the cavity of the drape via the proximal opening of the drape.

5. A method of draping a robotic surgical system, comprising:
positioning a drape, in a folded form, adjacent the robotic surgical system, the drape including:
a body having a distal opening, a proximal opening, and a cavity extending along a length of the body, the distal opening including a rigid ring and the proximal opening including a proximal edge; and
a deployment handle disposed on the body at a position distal of the proximal edge of the proximal opening; and
locating a surgical assembly of the robotic surgical system within the proximal opening of the drape via the deployment handle such that the surgical assembly is disposed within the cavity of the body adjacent the proximal opening.

6. The method according to claim 5, further comprising:
advancing the drape, in the folded form, over a slide rail and an instrument drive unit of the surgical assembly until a distal end of the instrument drive unit is aligned with the rigid ring of the drape; and
fixing the distal end of the instrument drive unit with the rigid ring of the drape.

7. The method according to claim 6, further comprising:
connecting a sterile interface module of the surgical assembly to the distal end of the instrument drive unit such that the rigid ring of the drape is disposed between the distal end of the instrument drive unit and the sterile interface module.

8. The method according to claim 7, further comprising:
maneuvering the drape proximally, via the deployment handle, such that the drape is telescopically unfolded from the folded form of the drape to a telescopically unfolded form of the drape, and a surgical robotic arm of the robotic surgical system is disposed within the cavity of the body of the drape.

9. The method according to claim 8, further comprising:
coupling a surgical instrument of the robotic surgical system with the sterile interface module of the surgical assembly.

10. A robotic surgical system, comprising:
a surgical robotic arm;
a surgical assembly coupled to the surgical robotic arm; and
a drape including:
a body having a distal opening, a proximal opening, and a cavity extending along a length of the body, the distal opening including a rigid ring and the proximal opening including a proximal edge, the cavity dimensioned for receipt of the surgical robotic arm and the surgical assembly via the proximal opening of the body; and
a deployment handle coupled to the body at a position distally adjacent of the proximal edge of the proximal opening, the deployment handle configured to facilitate positioning of the body of the drape adjacent the robotic surgical system such that the surgical robotic arm and the surgical assembly of the robotic surgical system is disposed within the cavity of the body.

11. The robotic surgical system according to claim 10, wherein the body of the drape has an outer surface and an inner surface, wherein the deployment handle is fixed to the outer surface of the body.

12. The robotic surgical system according to claim 11, wherein the body includes a first end portion and a second end portion, the second end portion having a proximal-most end, wherein the deployment handle is fixed to the outer surface of the body, distally adjacent the proximal-most end of the body.

13. The robotic surgical system according to claim 11, wherein the deployment handle includes a body having a first portion and a second portion spaced apart and coupled to the outer surface of the body such that a handle loop is formed therebetween, the handle loop dimensioned to receive a hand of a clinician to facilitate positioning of the surgical robotic arm and the surgical assembly of the robotic surgical system within the cavity of the drape via the proximal opening of the drape.

14. A surgical drape system for covering a robotic surgical system, comprising:
a first drape including:
a body including a first end portion and a second end portion, the body having a cavity extending between the first end portion and the second end portion thereof, the cavity dimensioned for receipt of a first portion of the robotic surgical system; and
a first deployment handle coupled to the second end portion of the body, the deployment handle configured to facilitate positioning of the body of the first drape adjacent the robotic surgical system such that the first portion of the robotic surgical system is disposed within the cavity of the body of the first drape;
a second drape including:
a body including a first end portion and a second end portion, the body having a cavity extending between the first end portion and the second end portion thereof, the cavity dimensioned for receipt of a second portion of the robotic surgical system; and
a second deployment handle coupled to the second end portion of the body, the deployment handle configured to facilitate positioning of the body of the second drape adjacent the robotic surgical system such that the second portion of the robotic surgical system is disposed within the cavity of the body of the first drape; and
a third drape including:
a body including a first end portion and a second end portion, the body having a cavity extending between the first end portion and the second end portion thereof, the cavity dimensioned for receipt of a third portion of the robotic surgical system.
